(19) United States
(12) Patent Application Publication
(10) Pub. No.: US 2026/0007304 A1
(43) Pub. Date: Jan. 8, 2026

(54) ENDOSCOPIC INSTRUMENT HAVING A HANDPIECE

(71) Applicant: Richard Wolf GmbH, Knittlingen (DE)

(72) Inventor: Sebastian FREY, Waghäusel (DE)

(21) Appl. No.: 18/880,906

(22) PCT Filed: Jul. 7, 2023
(86) PCT No.: PCT/DE2023/200138

§ 371 Date: Jan. 3, 2025

(30) Foreign Application Priority Data
Jul. 7, 2022 (DE) 10 2022 206 945.5

(51) Int. Cl. A61B 1/00

(52) U.S. Cl. CPC A61B 1/00121; A61B 1/00128

(57) ABSTRACT

An endoscopic instrument with a hand-piece (2), a shank which coming from the hand- piece (2) extends distally and an interface element (8) which is arranged on the proximal end of the hand-piece (2) and which is connected to the hand-piece (2) via a releasable coupling device (10, 12), wherein the coupling device (10, 12) comprises a magnet arrangement with at least one permanent magnet (14, 16) which effects an attraction force between the interface element (8) and the hand-piece (2).

### CROSS REFERENCE TO RELATED APPLICATIONS

This application is a United States National Phase Application of International Application PCT/DE2023/200138, filed Jul. 7, 2023, and claims the benefit of priority under 35 U.S.C. § 119 of German Application DE 10 2022 206 945.5, filed Jul. 7, 2022, the entire contents of which are incorporated herein by reference.

### TECHNICAL FIELD

The invention relates to an endoscopic instrument with a hand-piece and with a releasable interface element.

### BACKGROUND

With regard to endoscopic instruments, it is known to provide releasable interface elements, for example exchangeable introduction funnels, on a hand-piece at the proximal end of a working channel. The interface elements on the one hand can be removable for cleaning purposes, but on the other hand however in particular can be exchangeable in order to be able to adapt the instrument to desired requirements.

### SUMMARY

It is the object of the invention to improve such an endoscopic instrument to the extent that a simple and reliable coupling between the hand-piece and the interface element is possible.

This object is achieved by an endoscopic instrument with features according to the invention. Preferred embodiments are disclosed in the description, the claims and the attached figures.

The endoscopic instrument according to the invention comprises a handle or hand-piece which serves for holding and handling the instrument. I.e. the hand-piece preferably has a shape which can be easily gripped, i.e. is adapted to the shape of the human hand. A shank which coming from the hand-piece extends distally is arranged on the hand-piece. The shank can be connected to the hand-piece in a fixed or releasable manner. An interface element which is connected to the hand-piece via a releasable coupling device is arranged on the proximal end of the hand-piece. The interface can be for example an introduction funnel which is arranged at the proximal end of a working channel. In such a case, the working channel preferably extends through the shank, connecting proximally through the hand-piece up to the introduction funnel. Herein, the interface element preferably lies on the shank axis at the opposite end of the hand-piece. The interface element is releasably connected to the hand-piece so that it can be removed from the hand-piece. For this, a releasable coupling device is provided between the hand-piece and the interface element. If the coupling device is released, then the interface element can be removed from the hand-piece. According to the invention, this coupling device comprises a magnet arrangement with at least one permanent magnet. The permanent magnet is herein arranged such that it effects an attraction force between the interface element and the hand-piece, i.e. holds the interface element on the hand-piece by magnetic attraction force. Such a magnet arrangement permits a simple connection between the interface element and the hand-piece, since these can automatically attract one another by way of permanent-magnetic force, preferably even before both have come into direct bearing contact with one another. Furthermore, the magnet arrangement also permits the locating of a predefined positioning between the interface element and hand-piece, in particular with respect to the angular position, in a simple manner since the magnetic fields can automatically align the interface element and the hand-piece on joining together. A coupling device which is easy to be positioned is therefore created. Moreover, permanent magnets can be embedded into the hand-piece and the interface element such that smooth as possible contact surfaces are created, such being able to be easily cleaned and preferably autoclaved.

The magnet arrangement is preferably arranged or aligned with respect to its magmatic fields such that the attraction force which acts between the interface element and the hand-piece is directed in the direction of, or along, a joining axis of the hand-piece and the interface element. The joining axis is herein that axis along which the interface element is moved onto the hand-piece in order to join both onto one another which is to say connect them to one another. In the case that the interface element is an introduction funnel at the end of the working channel, the joining axis preferably runs in the longitudinal direction of the working channel, further preferably parallel to the longitudinal axis of the shank which is arranged at the distal end of the hand-piece.

Further preferably, the magnet arrangement is designed such that it sets at least a defined angular position between the hand-piece and the interface element with respect to their joining axis, as a magnetic latching position. This can be achieved for example by way of the magnet arrangement exerting particularly high forces of attraction in at least one certain angular position and exerting weaker or no attraction forces at all in other angular positions. This is achieved by a pointwise arrangement of the magnet poles or of the magnet arrangement in the at least one defined angular position, wherein preferably corresponding attraction forces then coming from the magnet poles also act in the circumferential region in a certain region and preferably automatically pull the interface element into the desired angular position on joining. Due to the alternating arrangement of magnet poles which are directed oppositely, this can yet be assisted further by way of repelling forces in undesirable angular positions.

The definition of magnetic latching positions compared to the definition of mechanical latching positions has the advantage that one can make do without a mechanical engagement and the contact surfaces do not need to comprise mechanical projections as latching elements or the like. Smooth contact surfaces can therefore be ensured and these are easier to clean and improve the hygienic design of the instrument.

The magnet arrangement preferably comprises at least one, further preferably several magnet pairs. The magnet pairs each comprise a first magnet pole on the hand-piece and an opposite second magnet pole on the interface element, wherein the first and the second magnet pole preferably lie opposite one another in a desired latching position or angular position. The first and the second magnet poles are preferably designed such that in a magnet pair, a first magnet pole is designed as a south pole and a second magnet pole as a north pole or vice versa, so that the first and the second magnet pole attract one another and muster the desired attraction or holding force.

Further preferably, the at least one magnet arrangement comprises several magnet pairs whose magnet poles are distributed, preferably uniformly distributed on a circumferential line about the joining axis. The magnet poles of the magnet pairs can therefore serve for defining the angular position of the interface element and the hand-piece with respect to the joining axis on joining, i.e. for setting the magnetic latching positions which are described above. The several magnet pairs are herein preferably distributed over the circumference such that the magnet poles of all magnets pairs lie opposite one another and are aligned in a manner attracting one another, in at least one desired angular position which corresponds to a desired joining position. By way of this, a particularly large attraction or holding force is realised. If the magnet pairs are distributed uniformly about the circumference, then further preferably a uniform introduction of force is ensured. Moreover, the desired joining position or angular position can thus be magnetically indicated in a particularly clear manner. If the poling of the magnets alternates over the circumference, then this would have the advantage that repulsion forces would arise given an incorrect, i.e. undesired angular position, such forces assisting in the moving of the elements into the desired angular position.

In a further preferred embodiment, the magnet arrangement comprises several magnet pairs, of which in each case all first magnet poles with their magnetic fields are aligned parallel to one another and all second magnet poles with their magnetic fields are aligned parallel to one another. In this manner, all magnet pairs can generate attraction forces in the same direction and it becomes possible to design the magnet pairs such that several possible joining positions are possible. Otherwise, the magnet pairs can be designed as has been described previously.

As described, the magnet pairs are preferably designed such that the first magnet poles and the second magnet poles are aligned attracting one another. In order to prevent a joining in undesirable angular positions, magnet poles however can also be aligned such that they repel one another. This can result in particular if several first and several second magnet poles are provided and these in their polarity are each arranged in a manner alternating with one another, as has already been described previously.

Apart from the magnet arrangement, the coupling device can additionally comprise at least one releasable mechanical locking which permits a non-positive and/or positive connection between the hand-piece and the interface element. I.e. the mechanical locking is designed such that in a locking position it creates the non-positive and/or positive connection and in a released position releases this connection, so that the coupling device is released and the interface element can be separated from the hand-piece. The mechanical locking can represent an additional securing which in particular creates a firmer connection since it is not so easy to release. The magnetic connection can be released by way of applying a force which exceeds the magnetic force, which is also necessary in order to be able to remove the interface element from the hand-piece. The additional mechanical locking is advantageous in order to secure a fixed connection given the operation. The mechanical locking is preferably designed such that it is only brought into engagement when the desired positioning between the interface element and the hand-piece is achieved by the magnet arrangement. I.e. preferably the magnetic coupling and the mechanical locking come into action in a successive manner. Firstly, preferably the interface element is fixed on the hand-piece in a desired position by way of the magnet arrangement and is subsequently reliably fixed by way of actuation of the mechanical locking. The mechanical locking can herein be for example a bayonet connection or screw connection which is brought into engagement, particularly preferably positive engagement by way of a movement of the locking element.

Thus the mechanical locking particularly preferably comprises a movable locking element which further preferably is movably mounted on the interface element. The locking element is herein movable such that it can be moved between a realised position and a locked position. The arrangement of the movable locking element on the interface element e.g. has the advantage that in the case of damage it can be easily exchanged together with the interface element, but that the rest of the endoscopic element, in particular the hand-piece can continue to be used. A further advantage lies in the hand-piece itself comprising fewer or preferably no moving elements and in this respect being simpler to clean.

According to a possible design, the locking element is designed as a ring element which is rotatably mounted about the joining axis. The ring element can be easily gripped and permits an intuitive handling.

Preferably, the magnet arrangement and further preferably the two magnet poles of the described magnet pairs are arranged in the movable locking element. This has the advantage that the locking element can be held in the desired positions by the magnet pairs and in turn can be secured by the magnetic forces. Hence the magnetic forces can fix the locking element in the released position and further preferably in the secured position by way of magnet forces. Moreover, the magnet pairs have the effect of the locking element being easily held in a defined position on joining the interface element and the hand-piece, i.e. preferably being held in the released position in which it does not inhibit the joining procedure. This can be ensured by the magnetic latching which is described above.

The locking preferably comprises a stationary engagement element and the locking element at least one engagement section. The engagement section can be brought into a positive engagement with the stationary engagement element by way of moving the locking element. In a first securing position of the locking element, the engagement section is in engagement with the engagement element and in a second released position of the locking element the engagement section is released from the engagement element. The locking element particularly preferably secures the securing element from separating in the direction of the jointing axis. Inasmuch as this is concerned, the engagement section and the engagement element are preferably designed as undercuts which engage behind one another in the direction of the joining axis and in the secured position ensure a positive engagement and a positive securing in the direction of the joining axis. In the released position, the engagement element and engagement section disengage such that they no longer engage behind one another in the joining direction and can be moved apart.

Particularly preferably, the magnet arrangement is arranged and positioned in the locking element such that it defines magnetic latching points for the locking element which preferably correspond at least to the securing and the released position. This has the advantage that the movement of the locking element provides haptic information to the user via the magnetic latching points and such information characterises the desired positions of the locking element. Moreover, the magnet forces have the effect of the locking element preferably being automatically pulled into the end position via the magnetic forces shortly before reaching the desired position. On joining together the interface element and the latching element, such a magnet arrangement furthermore preferably ensures that the locking element is firstly held in the released position which is to say comes into contact with the counter-side, for example the hand-piece and can only be moved into the secured position by way of subsequent movement.

Particularly preferably, as has already described previously the locking element is arranged in a rotatable manner and the released and the securing position form defined, preferably equally large angular steps which alternate in the circumferential direction. The angular steps can be for example 90°, so that by way of rotation about 90° the locking element is moved from the released into the locked position. If the locking element is freely rotatable in the circumferential direction, then two released and two secured angular positions can result, and these alternate by 90°, so that the two secured angular positions are positioned lying 180° opposite one another and the two released angular positions are likewise positioned lying 180° opposite one another. The rotation is herein preferably effected about the joining axis.

The invention is hereinafter described by way of example and by way of the attached figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side view showing an example for an endoscopic instrument according to the invention;

FIG. 2 is a perspective detailed view of the proximal end of the hand-piece of the instrument according to FIG. 1 with a removed interface element;

FIG. 3 is a schematic sectioned view of the interface element and the hand-piece in the released state;

FIG. 4 is a schematic sectioned view of the hand-piece with the interface element in the state in which they are joined together;

FIG. 5 is a schematic sectioned view showing the arrangement of the hand-piece and the interface element according to FIG. 4 in the locked state;

FIG. 6 is a plan view of the coupling region on the hand-piece; and

FIG. 7 is a plan view of the coupling region on the interface element.

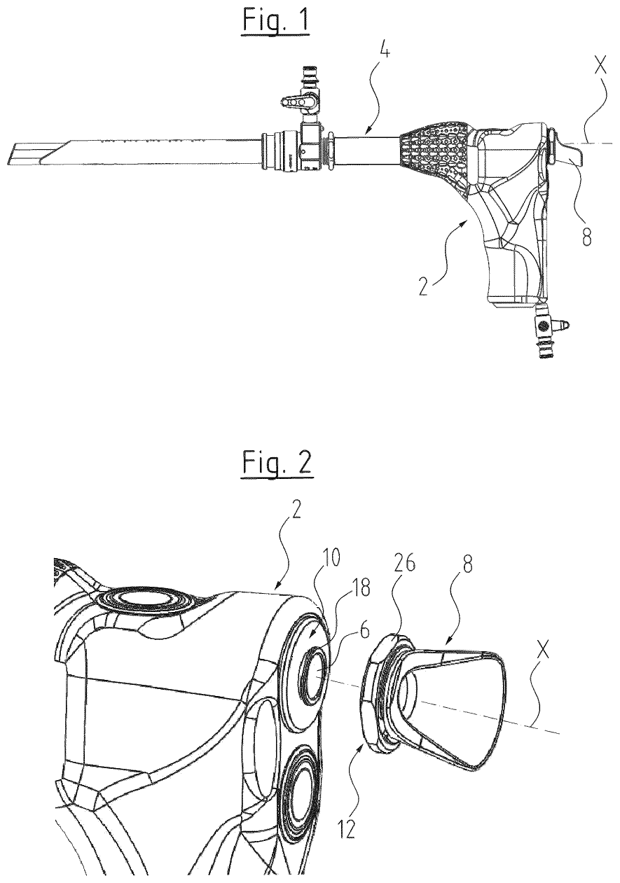

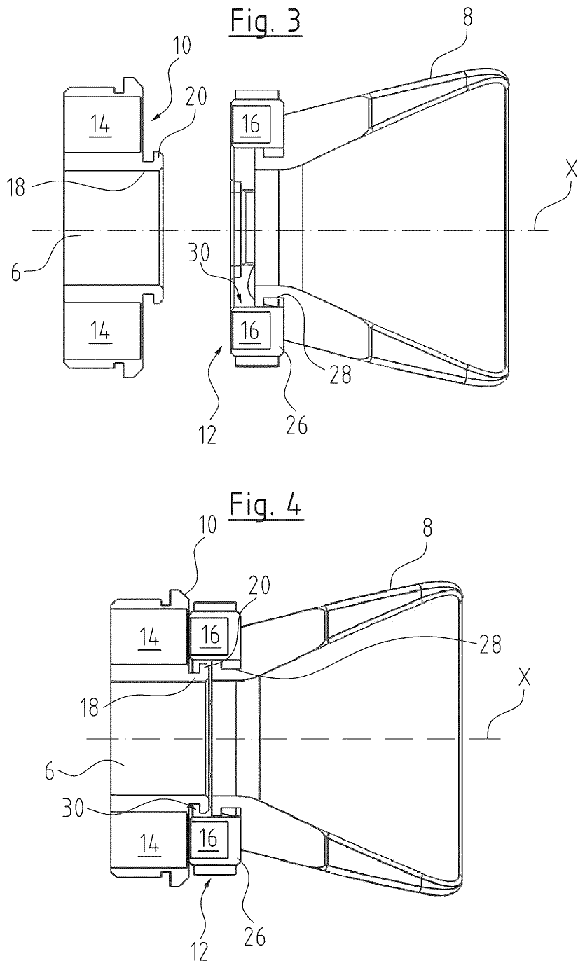

### DESCRIPTION OF PREFERRED EMBODIMENTS

Referring to the drawings, the endoscopic instrument which is shown as an example comprises a handle or a hand-piece 2 which serves for holding and handling the instrument. Coming from the hand-piece 2, a shank 4 extends distally and in the known manner can comprise at least one working channel as well as suction and/or rinsing channels. Moreover, the shank 4 in the known manner can comprise endoscope optics or a camera system as well as illumination elements.

A working channel in the shank 4 is open towards the proximal end of the hand-piece 2 so that a receiving opening 6 is situated at the proximal end, through which opening instruments can be advanced into the working channel and up to the distal end. An introduction funnel 8 which simplifies the introduction of instruments is applied onto the receiving opening 6. The introduction funnel forms an exchangeable interface element. I.e. differently shaped introduction channels 8 can be provided and these can be selectively coupled to the hand-piece 2.

In order to realise this coupling or exchangeability, a coupling device is formed between the hand-piece 2 and the introduction funnel 8. The coupling device comprises a first coupling part 10 which concentrically surrounds the receiving opening 6. A complementary second coupling part 12 is formed on the opposite side of the introduction funnel 8.

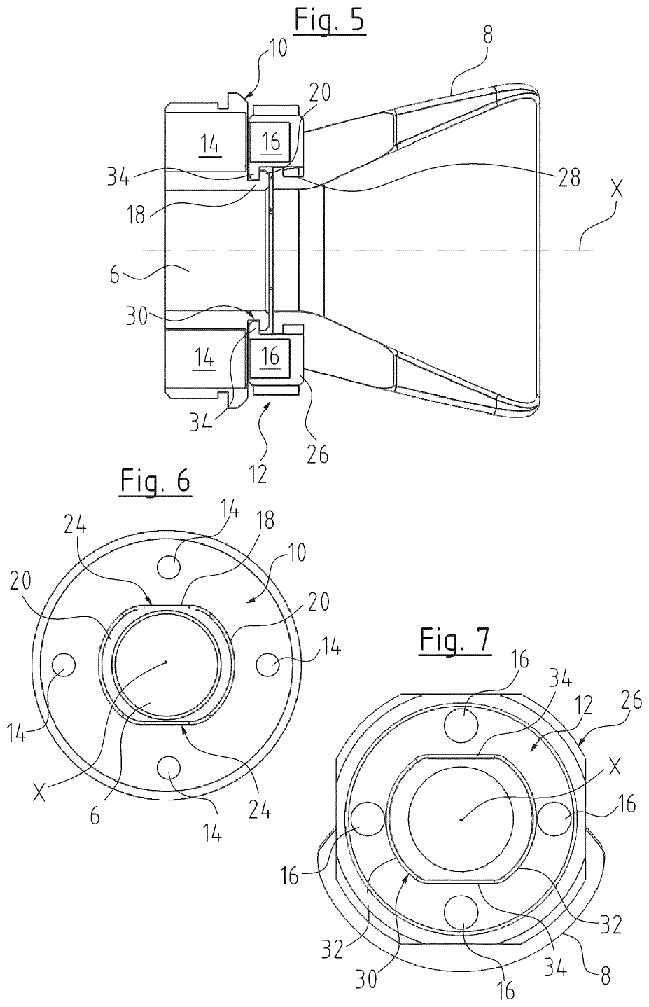

The coupling device comprises two essential coupling elements or coupling structures, specifically on the one hand a magnetic coupling and on the other hand a mechanical locking system. FIG. 6 shows a plan view of the first coupling part 10 which concentrically surrounds the receiving opening 6 or the joining axis X. The joining axis X which in this case corresponds to the longitudinal axis of the working channel 6 is that direction in which the introduction funnel 6 is applied or joined onto the hand-piece 2. Four magnets 14 are arranged in the first coupling part 10 or embedded into the surface, in a manner concentrically surrounding this joining axis X. Herein, the magnets can also be covered by a housing wall. In the shown example, the magnets are 14 designed as embedded permanent magnets. The magnets 14 are each arranged at the same radial distance to the joining axis X and are each positioned distanced to one another by 90° in the circumferential direction. The second coupling part 12 on the introduction funnel 8 comprises four complementary magnets 16. Likewise, four permanent magnets 16 are provided and these are radially distanced to the joining axis X essentially by the same amount and are likewise arranged in angular positions distanced to one another by 90°. Thus the magnets 14 and 16 can come into contact with one another or be arranged lying opposite one another, on joining the introduction funnel 8 onto the hand-piece 2. Herein, the magnets 14 form first magnet poles whilst the magnets 16 form second magnet poles. The magnets 14 and 16 are preferably aligned such that their magnetic axes, i.e. the axes between the north pole and south pole run parallel to the joining axis X, wherein the magnets 14 and 16 are arranged lying opposite one another such that either a north pole of a magnet 16 lies opposite a south pole of the magnet 14 or vice versa. I.e. the magnets 14 and 16 are equally directed in their polarity, so that they attract one another on joining together.

The magnets 14 and 16 one the one hand have the effect of introduction funnel 8 being pulled onto the hand-piece 2 in the axial direction, i.e. parallel to the joining axis X and being able to be magnetically held on this. Furthermore, magnetic latching points are created by the pointwise arrangement of the magnets 14 and 16 at four angular positions and these points ensure that the introduction funnel 8 can be applied onto the hand-piece 2 or its first coupling part 10 in defined angular positions. In this case these are four possible angular positions which are defined by the positions of the magnets 14 and 16.

The magnetic holding force provides a first fixation of the introduction funnel 8 to the hand-piece 2. For securing, a mechanical securement is additionally provided and is designed as follows. Surrounding the receiving opening 6, the first coupling part 10 comprises a sleeve 18 which projects in the proximal direction and which at its proximal end comprises a radially outwardly directed shoulder 20. The shoulder 20 forms a collar-like radially outwardly directed projection which however does not extend over the complete periphery but merely at two diametrically opposite sides with respect to the joining axis X. The otherwise annular shoulder 20 is sectioned or flattened in the direction of a chord in two sections 24 between these two sides, as can be recognised in FIG. 6.

A movable locking element in the form of a rotatable locking ring 26 is arranged or mounted on the introduction funnel 8 as an exchangeable interface element. The locking ring 26 extends concentrically about the joining axis X and is rotatably mounted via an engagement into a groove 28 on the introduction funnel 8. The magnets 16 which form the second magnet poles are arranged or embedded in the locking ring 26. I.e. the magnets 16 are rotatable together with the locking ring 26 about the joining axis X. The locking ring 26 in its inner region comprises a receiver 30 which in its geometry is adapted to the geometry of the sleeve 18 with the shoulder 20. I.e. the receiver 30 comprises two arcuate sections 32 which are arranged lying diametrically opposite with respect to the joining axis X and comprise an arc which is slightly larger than the outer circumference of the shoulder 20. Straight or chord-like sections 34 which extend parallel to the sections 24 on the sleeve 18 and are distanced somewhat further from one another than the sections 24 are situated between the arcuate sections 32. The straight sections 34 form engagement sections which can come into engagement with engagement elements which are formed by the shoulder 20.

In a first angular position or magnetic latching position which can be defined by the magnets 14 and 16, the receiver 30 can be placed onto the sleeve 30 with the shoulder 20, wherein the straight sections 34 lie parallel to the sections 24 and thus the sleeve 18 with the shoulder 20 can enter into the receiver 30. This is shown in FIG. 4. The locking ring 26 is subsequently rotated by 90°. Herein, firstly the holding force between the magnets 14 and 16 must be overcome. The locking ring 36 is herewith rotated out of a first magnetic latching position. If the magnets 16 are removed from the magnets 14, then the holding force reduces until the magnets 16 can come into the proximity of the adjacent magnets 14 in the circumferential direction. The holding force then increases again and the locking ring 26 rotates into the next latching position which is offset by 90°. In this position, the straight sections 34 of the receiver 30 now engage behind the shoulder 20 and herewith form a positive engagement in the direction of the joining axis X, so that the introduction funnel 8 is held on the first coupling part 10 in a positive manner in the direction of the joining axis X. For releasing, the locking ring 36 is again rotated by 90° by which means the sections 34 of the receiver 30 disengage again from the shoulder 20 on the sleeve 18 and are then again situated parallel to the straight sections 24 on the sleeve 18. In this angular position, the introduction funnel 8 can be pulled from the hand-piece 2 after overcoming the magnetic holding force between the magnets 14 and 16.

It is to be understood that instead of an introduction funnel 8, another exchangeable interface element could be coupled to the hand-piece 2 in a similar manner, e.g. a hose coupling or lead coupling, connection plug etc. Herein, the coupling comprises an arrangement of permanent magnets which creates a magnetic holding force between the interface element and the hand-piece 2. Further preferably, a second mechanical securing is provided, and apart from the magnetic coupling this is realised for securing a positive engagement between the interface element and the hand-piece 2. Herein, the magnetic coupling and the mechanical securing are preferably designed such that they can be successively brought into engagement, i.e. the magnetic coupling permits a first fixation and subsequently the mechanical locking with the positive engagement can take place.

While specific embodiments of the invention have been shown and described in detail to illustrate the application of the principles of the invention, it will be understood that the invention may be embodied otherwise without departing from such principles.

### LIST OF REFERENCE NUMERALS

2—hand-piece
4—shank
6—receiving opening
8—introduction funnel
10—first coupling part
12—second coupling part
14—magnets
16—magnets
18—sleeve
20—shoulder, engagement elements
24—straight sections
26—locking ring
28—groove
30—receiver
32—arcuate sections
34—straight sections, engagement sections
X—joining axis, longitudinal axis

1. An endoscopic instrument comprising:
a hand-piece;,
a shank which coming from the hand-piece extends distally;
an interface element which is arranged on a proximal end of the hand-piece and
a releasable coupling device, wherein the interface element is connected to the hand-piece via the coupling device, and wherein the coupling device comprises a magnet arrangement with at least one permanent magnet which effects an attraction force between the interface element and the hand-piece.

2. An endoscopic instrument according to claim 1, wherein the at least one magnet arrangement is arranged such that the attraction force is directed along a joining axis of the hand-piece and the interface element.

3. An endoscopic instrument according to claim 1, wherein the at least one magnet arrangement is configured to set at least one defined angular position between the hand-piece and the interface element with respect to a hand-piece and interface element joining axis, as a magnetic latching position.

4. An endoscopic instrument according to claim 1, wherein the magnet arrangement comprises at least one, magnet pairs which has a first magnet pole on the hand-piece and an opposite second magnet pole on the interface element.

5. An endoscopic instrument according to claim 1, wherein the at least one magnet arrangement comprises several magnet pairs whose magnet poles are distributed on a circumferential line about the joining axis.

6. An endoscopic instrument according to claim 1, wherein the at least one magnet arrangement comprises several magnet pairs, of which all first magnet poles with first pole magnetic fields are aligned parallel to one another and all second magnet poles with second pole magnetic fields are aligned parallel to one another.

7. An endoscopic instrument according to claim 6, wherein the first magnet poles and the second magnet poles are aligned in the magnet pairs in a manner attracting one another.

8. An endoscopic instrument according to claim 1, wherein the coupling device additionally comprises at least one releasable mechanical locking which permits a non-positive and/or positive connection between the hand-piece and the interface element.

9. An endoscopic instrument according to claim 8, wherein the mechanical locking comprises a movable locking element.

10. An endoscopic instrument according to claim 9, wherein the locking element is configured as a ring element which is rotatably mounted about the joining axis.

11. An endoscopic instrument according to claim 8, wherein the at least one magnet arrangement and the second magnet poles of the magnet pairs are arranged in the movable locking element.

12. An endoscopic instrument according to claim 8, wherein the locking comprises at least one stationary engagement element and the locking element comprises at least one engagement section which in a first, securing position of the locking element, is in engagement with the at least one engagement element and in a second, released position of the locking element, is released from the engagement element.

13. An endoscopic instrument according to claim 12, wherein the magnet arrangement is arranged in the locking element in a manner such that the magnet arrangement defines magnetic latching points for the locking element.

14. An endoscopic instrument according to claim 13, wherein the locking element (26) is rotatable and released and securing positions alternate as defined, preferably equally large angular steps.

15. An endoscopic instrument according to claim 1, wherein the interface element is an introduction funnel at the proximal end of a working channel.

16. An endoscopic instrument according to claim 5, wherein the at least one magnet arrangement comprises several magnet pairs whose magnet poles are distributed in a uniform manner.

17. An endoscopic instrument according to claim 7, wherein the mechanical locking comprises a movable locking element which is movably mounted on the interface element.

18. An endoscopic instrument according to claim 13, wherein the magnet arrangement is arranged in the locking element such that the magnet arrangement defines magnetic latching points for the locking element in the securing and the released position.

19. An endoscopic instrument according to claim 14, wherein the locking element is rotatable and released and securing positions alternate as equally large angular steps.
